(19) United States
(12) Patent Application Publication
(10) Pub. No.: US 2012/0142789 A1
(43) Pub. Date: Jun. 7, 2012

(54) Catalytic Reaction Module

(75) Inventors: Ross Alexander Morgan, Oxfordshire (GB); Robert Peat, Oxfordshire (GB); Tuan Quoc Ly, Oxfordshire (GB)

(73) Assignee: Compact GTL plc, Cleveland (GB)

(21) Appl. No.: 13/388,160

(22) PCT Filed: Aug. 6, 2010
(86) PCT No.: PCT/GB2010/051309

§ 371 Date: Jan. 31, 2012

(30) Foreign Application Priority Data
Aug. 28, 2009 (GB) 0915036.8

(51) Int. Cl. C07C 1/04; B01J 8/02

(52) U.S. Cl. 518/702; 422/629

(57) ABSTRACT

A catalytic reaction module for performing am endothermic reaction, such as steam reforming, including separator blocks. Each reactor defining a multiplicity of first and second flow channels arranged alternately within the block to ensure thermal contact between the first and second flow channels. The reactor blocks may be arranged and connected for series flow of a combustible gas mixture in the first flow channels. The reactor blocks may be arranged and connected for a gas mixture to undergo endothermic reaction in the second flow channels. Catalyst elements are provided within the flow channels and the catalyst may vary between the blocks or within a block. The catalyst may vary in chemical composition, in catalyst loading, or in active catalyst material.

This invention relates to a catalytic reaction module with channels for performing an endothermic chemical reaction such as steam reforming, in which the heat is provided by a combustion reaction in adjacent channels, and to a method for performing an endothermic chemical reaction with such a module.

A plant and process are described in WO 2005/102511 (GTL Microsystems AG) in which methane is reacted with steam, to generate carbon monoxide and hydrogen in a first catalytic reactor; the resulting gas mixture is then used to perform Fischer-Tropsch synthesis in a second catalytic reactor. The reforming reaction is typically carried out at a temperature of about 800° C., and the heat required may be provided by catalytic combustion in channels adjacent to those in which reforming is carried out, the combustion channels containing a catalyst which may comprise palladium or palladium/platinum on an alumina support in the form of a thin coating on a metallic substrate. An inflammable gas mixture such as a mixture of methane and air is supplied to the combustion channels. Combustion occurs at the surface of the catalyst without a flame. However, it has been found that the combustion reaction tends to occur most vigorously near the start of the combustion channel, which can lead to an unsuitable temperature distribution along the channel; although this problem may be overcome by staging fuel injection along the combustion channel, an alternative solution would be desirable.

According to the present invention there is provided a catalytic reaction module for performing an endothermic reaction, the module comprising a plurality of separate reactor blocks, each reactor block defining a multiplicity of first and second flow channels arranged alternately within the block to ensure thermal contact between the first and second flow channels, with catalyst in the first flow channels for the endothermic reaction, and with catalyst in the second flow channels for a combustion reaction, the reactor blocks being arranged and connected for series flow of a gas mixture to undergo the endothermic reaction in the first flow channels and also for flow of a combustible gas mixture in the second flow channels, such that the endothermic reaction mixture flows in series through the reactor block, wherein in the first flow channels and/or the second flow channels the respective catalyst varies between one reactor block and another, and/or between one part of a reactor block and another.

The reactor blocks are referred to as being separate in the sense that they have distinct and separate inlets and outlets for the gas mixtures. The reactor blocks may also be physically separate, that is to say spaced apart from each other; or they may be joined together for example as a stack.

Preferably the module is arranged such that the combustible gas mixture provided to a reactor block is at an elevated temperature below its auto-ignition temperature, the temperature being raised at least in part as a result of combustion of combustible gas mixture in one or more of the reactor blocks. Indeed, preferably, the combustible gas mixture provided to each reactor block in the module is at such an elevated temperature. For at least some of the blocks the temperature may be raised by heat exchange with gases emerging from the second gas flow channels of one or more of the reactor blocks. In one preferred embodiment the combustible gas mixture is arranged to flow in series through the reactor blocks in the same order as the endothermic gas mixture. In this case the combustible gas mixture provided to a second or subsequent reactor block is at an elevated temperature as a result of having at least partly undergone combustion in the preceding reactor block of the series.

The combustible gas mixture comprises a fuel (such as methane) and a source of oxygen (such as air). In one example, the combustible gas mixture flows through the reactor blocks in series. The outflowing gases from the combustion channels of the first reactor block may be introduced directly into the second reactor block without modification or treatment, so the module acts as if it were a single stage with reactor channels longer than those of a single reactor block. Alternatively, it may be that between successive reactor blocks means are provided to treat the outflowing gas mixture that has undergone combustion, for example to change its temperature, or to introduce and mix in additional or different fuel. It may also be desirable between successive reactor blocks to provide means to introduce additional air into the outflowing gas mixture that results from combustion. By providing a module in which the provision of fuel can be staged between different reactor blocks and in which the introduction of air can be staged, greater control over the temperature distribution can be achieved. For example, if there are two reactor blocks in series, the proportion of the fuel provided at the first stage is preferably between 50% and 70% of the total required fuel, the remainder being provided for the second stage.

The invention also provides a method of performing an endothermic reaction wherein the heat required for the endothermic reaction is provided by a combustion reaction in an adjacent channel to the endothermic reaction, wherein the endothermic reaction is carried out in a plurality of successive stages. The endothermic reaction may be steam methane reforming, and in this case preferably the temperature in the endothermic reaction channels increases through the first stage to between 675° C. and 700° C., preferably to about 690° C.; and increases through the second stage to between 730° C. and 800° C., preferably to about 770° C. In a preferred embodiment the combustion reaction is also carried out in at least two successive stages, with treatment of the combustion gas mixture emerging from one stage before it is introduced to the next stage.

It will be appreciated that there are some treatments that may be applied either to a combustion gas mixture emerging from one stage before it is introduced to a subsequent stage, or alternatively may be applied to a combustion gas mixture before it is introduced into a reactor module, whether the reactor module operates as if it were a single stage, or as more than one stage. Such treatment may include the introduction of an inert component into the gas mixture. This inert component may be for example steam and/or carbon dioxide, or may be nitrogen; a steam/carbon dioxide mixture may be obtained from the product gases. The provision of such an inert component within the combustion gas mixture helps to suppress the rate of combustion, because it reduces the partial pressures of the reactants, i.e. the oxygen and the fuel. Where the inert component is steam or carbon dioxide, this adsorbs onto the surface of the catalyst, so further suppressing the rate of combustion.

The combustion catalyst may comprise palladium oxide, which is stable at room temperature, and an active catalyst. At temperatures above about 600° C. the catalyst gradually converts to a mixture of palladium and palladium oxide, at a rate that depends on the partial pressure of oxygen to which it is exposed. This conversion therefore occurs during operation, over the first few days of operation. Palladium is a less active catalyst than palladium oxide, so the catalytic activity gradually decreases over the first few days of operation of the reactor module, before reaching a stable value. The addition of inert components into the combustion gas mixture ensures that this decrease of initial activity and stabilisation of catalytic activity is brought about more rapidly. For example stable operation can be attained within about 30 hours, rather than about 80 hours.

The addition of inert gases, such as combustion exhaust gases, to the combustion gas mixture not only enables stable operation to be achieved more rapidly, but enables stable operation to be ensured during prolonged operation. For example, in a module in which no treatment is provided to the combustion gas mixture between successive reactor blocks, the required quantity of fuel, along with air, must be applied to the inlet to the module. If exhaust gas is also added to the combustion gas mixture supplied to the inlet of the module it suppresses the rate of reaction. The quantity of added exhaust gas can be adjusted in accordance with the activity of the combustion catalyst, to achieve a desired temperature distribution and reaction rate, and so a desired conversion achieved by the endothermic reaction. If the activity of the combustion catalyst decreases during operation, for example over a period of months or years, the proportion of exhaust gas can be decreased so as to maintain the desired temperature distribution and reaction rate. This technique is also applicable if the combustion catalyst is initially more active than required, as the initial activity can be suppressed by the added exhaust gas. If the catalyst degrades over its life to such an extent that no exhaust gas need be added, the combustion reaction may then be enhanced by adding additional fuel. Eventually the combustion catalyst may have to be replaced.

Over the life of the reactor module the catalysts will tend to degrade, and it may be desirable to increase the temperature to which the gas mixtures are preheated before they are fed into the reactor blocks so as to counteract the decrease in activity of the catalyst. For the second stage combustion channels it may be desirable to introduce an oxygen-rich gas into the combustible gas stream, so as to raise the partial pressure of oxygen; although this may be necessary throughout the operation of the reactor module, it is particularly desirable as the catalyst degrades. Furthermore the pressure within the combustion channels may also be increased. This pressure increase generally increases the rate of the combustion reaction, and may therefore be advantageous to maintain activity as the combustion catalyst degrades. Not only can the fuel/air ratio differ between the first and second stage reactor blocks, but the combustible component may also be varied, for example it may be desirable to use a gas mixture with a higher hydrogen partial pressure for the second stage than for the first stage.

Where there is treatment of the combustion gas mixture between successive stages, this treatment preferably comprises changing its temperature and adding additional fuel. By lowering the gas temperature before adding additional fuel, auto-ignition can be avoided.

By performing the combustion process in a number of stages, using separate reactor blocks, the benefits of staged fuel injection are obtained—for example a more uniform temperature distribution along the reactor module—while avoiding potential problems. In particular this makes it possible to cool the combustion gas mixture between successive stages, before introducing additional fuel, which can ensure that auto-ignition does not occur. The treatment of the combustion gas mixture between successive reactor blocks takes place within the module, but not within the reactor blocks.

Preferably the first flow channels and the second flow channels extend in parallel directions, within a reactor block, and the combustible gas mixture and the endothermic reaction mixture flow in the same direction (co-flow). Preferably the flow channels are of length at least 300 mm, more preferably at least 500 mm, but preferably no longer than 1000 mm. A preferred length is between 500 mm and 700 mm, for example 600 mm. It has been found that co-flow operation gives better temperature control, and less risk of hot-spots.

In the preferred embodiment each first flow channel (the channels for the endothermic reaction) and each second flow channel (the channels for the combustion reaction) contains a removable catalyst structure to catalyse the respective reaction, each catalyst structure preferably comprising a metal substrate, and incorporating an appropriate catalytic material. Each such catalyst structure should be non-structural, in that it does not provide any mechanical support to the walls of the flow channel. Preferably each such catalyst structure is shaped so as to subdivide the flow channel into a multiplicity of flow sub-channels. The flow sub-channels may be straight and parallel, or alternatively the flow sub-channels in a single layer may be parallel to one another, but have a herringbone or other similar pattern so that the sub-channels in one layer are not parallel to the sub-channels in the layer above or below. Preferably each catalyst structure includes a ceramic support material on the metal substrate, which provides a support for the catalyst.

The metal substrate provides strength to the catalyst structure and enhances thermal transfer by conduction. Preferably the metal substrate is of a steel alloy that forms an adherent surface coating of aluminium oxide when heated, for example a ferritic steel alloy that incorporates aluminium (eg Fecralloy™). The substrate may be a foil, a wire mesh, expanded foam, or a felt sheet, which may be corrugated, dimpled or pleated; the preferred substrate is a thin metal foil for example of thickness less than 100 μm, which is corrugated to define the sub-channels.

Each reactor block may comprise a stack of plates. For example, the first and second flow channels may be defined by grooves in respective plates, the plates being stacked and then bonded together. Alternatively the flow channels may be defined by thin metal sheets that are castellated, i.e. formed into rectangular corrugations, and stacked alternately with flat sheets; the edges of the flow channels may be defined by sealing strips. To ensure the required good thermal contact both the first and the second gas flow channels may be between 10 mm and 2 mm high (in cross-section); and each channel may be of width between about 3 mm and 25 mm. The stack of plates forming the reactor block is bonded together for example by diffusion bonding, brazing, or hot isostatic pressing.

Preferably a flame arrestor is provided at the inlet to each flow channel for combustion to ensure a flame cannot propagate back into the combustible gas mixture being fed to the combustion channel. This may be within an inlet part of each combustion channel, for example in the form of a non-catalytic insert that subdivides a portion of the combustion channel adjacent to the inlet into a multiplicity of narrow flow paths which are no wider than the maximum gap size for preventing flame propagation. For example such a non-catalytic insert may be a longitudinally-corrugated foil or a plurality of longitudinally-corrugated foils in a stack. Alternatively or additionally, where the combustible gas is supplied through a header, then such a flame arrestor may be provided within the header.

The invention will now be further and more particularly described, by way of example only, and with reference to the accompanying drawings, in which:

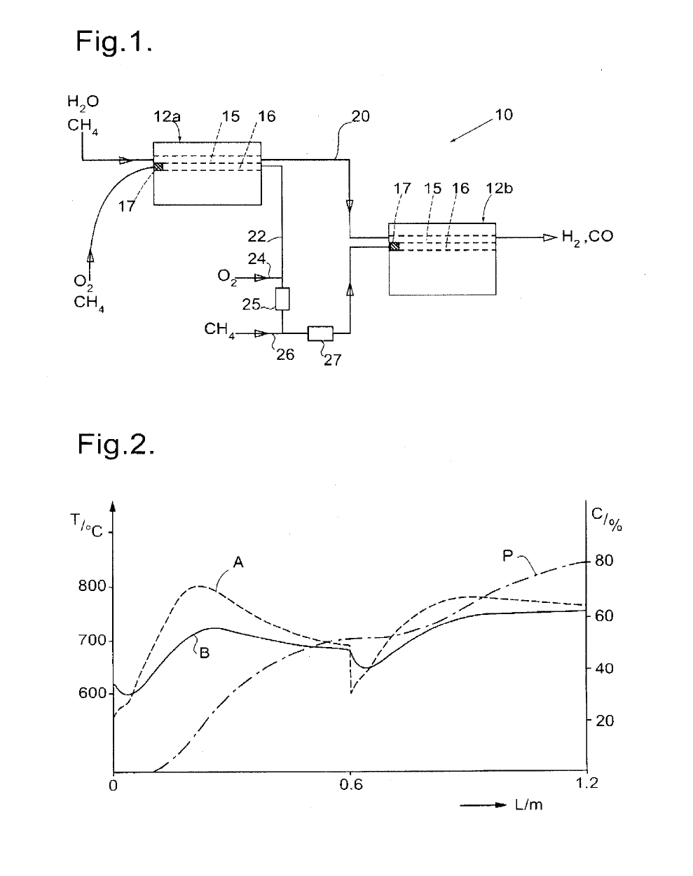

FIG. 1 shows a diagrammatic side view of a reaction module of the invention; and

FIG. 2 shows graphically the variation of temperature through the reactor module of FIG. 1, and the corresponding variation of conversion in the steam methane reaction.

The steam reforming reaction of methane is brought about by mixing steam and methane, and contacting the mixture with a suitable catalyst at an elevated temperature so the steam and methane react to form carbon monoxide and hydrogen (which may be referred to as synthesis gas or syngas). The steam reforming reaction is endothermic, and the heat is provided by catalytic combustion, for example of methane mixed with air. The combustion takes place over a combustion catalyst within adjacent flow channels within a reforming reactor. Preferably the steam/methane mixture is preheated, for example to over 600° C., before being introduced into the reactor. The temperature in the reformer reactor therefore typically increases from about 600° C. at the inlet to about 750-800° C. at the outlet.

The total quantity of combustion fuel (e.g. methane) that is required is that needed to provide the heat for the endothermic reaction, and for the temperature increase of the gases (sensible heat), and for any heat loss to the environment; the quantity of air required is up to 10% more than that needed to react with that amount of fuel.

Referring now to FIG. 1 there is shown a reaction module 10 suitable for use as a steam reforming reactor. The reaction module 10 consists of two reactor blocks 12a and 12b each of which consists of a stack of plates that are rectangular in plan view, each plate being of corrosion resistant high-temperature alloy. Flat plates are arranged alternately with castellated plates so as to define straight-through channels between opposite ends of the stack, each channel having an active part of length 600 mm. By way of illustration, the height of the castellations (typically in the range 2-10 mm) might be 3 mm in a first example, or might be 10 mm in a second example, while the wavelength of the castellations might be such that successive ligaments are 20 mm apart in the first example or might be 3 mm apart in the second example. All the channels extend parallel to each other, there being headers so that a steam/methane mixture can be provided to a first set of channels 15 and an air/methane mixture provided to a second set of channels 16, the first and the second channels alternating in the stack (the channels 15 and 16 being represented diagrammatically). Appropriate catalysts for the respective reactions are provided on corrugated foils (not shown) in the active parts of the channels 15 and 16, so that the void fraction is about 0.9. A flame arrestor 17 is provided at the inlet of each of the combustion channels 16. The flow channel at the ends of the stack, that is to say at the top and the bottom of the stack, might be one of the second set of channels 16, but alternatively it might be one of the first set of channels 15.

By way of example there may be over fifty such castellated plates in each stack.

The steam/methane mixture flows through the reactor blocks 12a and 12b in series, there being a duct 20 connecting the outlet from the channels 15 of the first reactor block 12a to the inlet of the channels 15 of the second reactor block 12b. Similarly the combustion mixture also flows through the reactor blocks 12a and 12b in series, there being a duct 22 connecting the outlet from the channels 16 of the first reactor block 12a to the inlet of the channels 16 of the second reactor block 12b. The duct 22 includes an inlet 24 for additional air, followed by a static mixer 25, and then an inlet 26 for additional fuel, followed by another static mixer 27.

In use of the reaction module 10, the steam/methane mixture is preheated to 620° C., and supplied to the reaction module 10 to flow through the reactor blocks 12a and 12b. A mixture of 80% of the required air and 60% of the required methane (as fuel) is preheated to 550° C., which is below the auto-ignition temperature for this composition, and is supplied to the first reactor block 12a. In both cases the preheating may be carried out by heat exchange with exhaust gases that have undergone combustion within the module 10. The temperature rises as a result of combustion at the catalyst, and the gases that result from this combustion emerge at a temperature of about 700° C. They are mixed with the remaining 20% of the required air (by the inlet 24 and the static mixer 25), and then with the remaining 40% of the required methane (by the inlet 26 and the static mixer 27), so that the gas mixture supplied to the combustion channels 16 of the second reactor block 12b is at about 600° C., which is again below the auto-ignition temperature for this mixture (which contains water vapour and carbon dioxide as a consequence of the first stage combustion). By adjusting the temperature of the additional air supplied at the inlet 24, the temperature of the resulting mixture can be controlled to be below the auto-ignition temperature.

By way of example the gas flow rates may be such that the space velocity is preferably between 14000 and 20000/hr and possibly more particularly between 15000 and 18000/hr (at a standard temperature and pressure of 15° C. and 1 atm) for the steam methane reforming channels (considering the reaction module 10 as a whole), and is preferably between 19000 and 23000/hr for the combustion channels (considering the reaction module 10 as a whole).

Referring now to FIG. 2, this shows graphically the variations in temperature T along the length L of the combustion channels 16 (marked A), and that along the reforming channels 15 (marked B). The portion of the graph between L=0 and L=0.6 m corresponds to the first reactor block 12a, while the portion of the graph between L=0.6 m and L=1.2 m corresponds to the second reactor block 12b. It will be noted that the temperature T in a reforming channel 15, once combustion has commenced, is always lower than the temperature T in the adjacent combustion channel 16. The combustion gas temperature undergoes a downward step change as a result of the added air (from inlet 24) between the first reactor block 12a and the second reactor block 12b (at position L=0.6 m). The variation of conversion of methane, C, in the steam reforming reaction with length L is shown by the graph marked P. The conversion increases continuously through the reaction module 10 and reaches a value of about 80%, which is close to the equilibrium conversion under the reaction conditions.

It will be understood that adjusting the space velocities in the combustion channels and in the reforming channels, and adjusting the proportion of fuel and of air provided for combustion to each reactor block, ensures that a satisfactory temperature distribution is achieved throughout the reactor blocks, and that thermal stresses within each reactor block are minimised. This ensures that the reactor module operates within safe margins, without risk of damage to the reactor blocks. It will also be appreciated that the variations in temperature and conversion shown in FIG. 2 are by way of example only, and that the temperature distribution and consequently the conversion will be slightly different for example if the combustion catalysts are altered or if the ratio of fuel to air is altered.

It will be appreciated that the description given above is by way of example only and that many changes may be made while remaining within the scope of the present invention. For example the dimensions of the channels 15 and 16 and of the reactor blocks 12 may differ from those indicated above. The proportions of air and methane supplied to the first reactor block 12a may differ from the proportions mentioned above. The proportion of fuel provided initially may be between 50% and 65%, more preferably 55% with the remaining 35% to 50%, preferably 45%, being provided between the blocks 12a and 12b. For example between 100% and 120% of the required air and 65% of the required fuel might be provided initially; and the remaining 35% of the fuel provided between the blocks 12a and 12b, although in that case it may be desirable to provide a heat exchanger (not shown) to cool the out-flowing gases to ensure the temperature is below the auto-ignition temperature. In every case the additional fuel is preferably added to a gas mixture that is below the auto-ignition temperature for the gas mixture under the prevalent conditions of gas composition and pressure. Where only part of the air is provided initially, as described above, this proportion is preferably at least 50%, and preferably no greater than 90%, more preferably between 75% and 85%, and most preferably 80% as in the example above. The quantity of air provided to a subsequent stage may be such that the total quantity of air exceeds 100% of that required, for example 80% may be provided at the first stage and 40% at the second stage. The added air introduces nitrogen which acts as an inert gas in this context.

It should be understood that the catalyst-carrying foils in the channels 15 and 16 preferably extend the entire length of the respective channels, apart from the initial part of the combustion channel 16 occupied by the flame arrestor 17. In a modification, no reforming catalyst is provided in an initial portion of each reforming channel 15, this initial non-catalytic portion being longer than the length of the flame arrestor 17, so that the gas mixture that is to undergo reforming is preheated before it reaches the reforming catalyst.

It should be appreciated that where the fuel gas consists of or contains a significant concentration (say >5%) of species such as H₂ and CO that have rapid combustion kinetics relative to methane, more than two reactor blocks and inter-stage mixing positions may be employed in order to control the temperature profile in the reactor module and prevent hot spots and adverse thermal gradients being generated.

The ability to modulate the proportions of fuel and air fed to each stage can also be used to compensate for reductions in catalyst activity over time. A further refinement with this arrangement is the ability to recycle some of the produced syngas to the fuel mixing stages to maintain the temperature profile in the reactor module as the combustion catalyst de-activates over time.

As will be appreciated, steam methane reforming may form part of a process for converting methane to longer-chain hydrocarbons, the synthesis gas produced by reforming then being subjected to Fischer-Tropsch synthesis. Alternatively, the synthesis gas may be subjected to a catalytic process to form methanol. The steam methane reforming in any such plant may be carried out using one or more reaction modules 10 as described above. A preferred plant incorporates several such reaction modules arranged in parallel, so that the plant capacity can be adjusted by changing the number of reaction modules that are utilised. If, for example, the synthesis gas is subjected to Fischer-Tropsch synthesis, the products will be water, longer chain hydrocarbons, and a tail gas containing hydrogen, carbon monoxide, and short chain hydrocarbons inter alia.

In the reaction module 10 shown in FIG. 1, and considering only the combustion channels 16, a platinum-palladium catalyst may be provided in both reactor blocks 12a and 12b. Alternatively the catalyst may be different in the two reactor blocks 12a and 12b. For example the catalyst in the first reactor block 12a may be platinum-palladium, and the catalyst in the second reactor block 12b instead might be platinum only. It will be appreciated that the oxygen partial pressure within the second reactor block 12b is less than that in the first reactor block 12a because of the combustion that has taken place. If a platinum-palladium catalyst is used in the second reactor block 12b a problem can arise, because this low oxygen partial pressure encourages the transformation of palladium oxide to palladium metal, and palladium metal is less effective as a combustion catalyst than palladium oxide. Hence there can be a benefit from using a platinum-only catalyst within the second reactor block 12b, or from using a platinum-palladium mixture with a high proportion of platinum in the second reactor block 12b. Platinum is catalytically active in the metal form, rather than the oxide form, and therefore the activity of the catalyst is not adversely affected by the low oxygen partial pressure within the second reactor block 12b. As another alternative a platinum-only catalyst could be used in both reactor blocks 12a and 12b. However, a platinum catalyst has a higher light-off temperature than a platinum-palladium catalyst, so it is not as suitable for use in the first reactor block 12a without the provision of additional heating at start-up, for example electrical heating. In addition, the oxygen partial pressure is higher in the first reactor block 12a and therefore the platinum-only catalyst does not provide the benefit that it would in the second reactor block 12b.

Not only may the active catalyst materials be different between the reactor blocks, or between different regions of a reactor block, but the catalyst loading (that is to say the ratio of ceramic support to foil) may be different. For example in the second reactor block 12b the quantity of ceramic (which incorporates the active catalytic material) might be as much as five times greater, more typically two times more, than in the first reactor block 12a. Furthermore the metal loading (that is to say the proportion of active catalytic material to the ceramic support) may differ between the first reactor block 12a and the second reactor block 12b. Furthermore the catalyst may vary along the length of a channel within a reactor block 12a or 12b. For example in the vicinity of the inlet to the combustion channels 16 the active catalytic material might be platinum-palladium, whereas further along the combustion channels 16 the active catalytic material might be platinum only, and the same arrangement of catalysts may apply within both the reactor blocks 12a and 12b. Equally the catalyst loading may vary along the length of a channel, and the metal loading may vary along the length of a channel. Any of these changes within a channel may be gradual along the length of the channel, but might instead be stepped. For example if the catalyst-carrying foils in the channels 16 extend the entire length of the channels then it may be convenient to have a gradual change of catalyst along the length of each foil, whereas if within each channel 16 there are two or three catalyst-carrying foils placed end to end then it may be convenient to have stepped changes of catalyst between one length of foil and the next.

As is evident from FIG. 2, particularly within the first reactor block 12a, the temperature tends to rise near the start of the channel as combustion is initiated. Some of the variations described above may therefore be applied in order to inhibit the rate of combustion near the start of the channel and so to reduce the rise in temperature.

Although the above description is in relation to the catalyst in the combustion channels 16, it will be appreciated that substantially the same variations may apply to the catalyst in the reforming channels 15. In this case, as long as the rate of heat transfer between the combustion channels 16 and the reforming channels 15 is not the limiting factor, the temperature rise near the start of the channels may also be inhibited by increasing the total quantity of the reforming catalyst near the start of the reforming channels 15 (by increasing the metal loading, and/or by increasing the catalyst loading), so as to increase the rate of the endothermic reforming reaction.

Where the channels are more than about 2 or 3 mm wide (in their narrowest transverse dimension) then it may be more convenient to provide the catalyst in a channel on a stack of corrugated foils separated by substantially flat foils, rather than on a single deep-formed corrugated foil. It will be appreciated that the nature of the catalyst on the flat foils may differ from that on the corrugated foils in the ways described above, that is to say differing in the nature of the active catalytic material, or in the catalyst loading, or in the active metal loading, or in more than one of these variables. Indeed the flat foils might carry no catalyst.

In particular it may be advantageous to provide an arrangement in which corrugated foils carrying a predominantly palladium-based catalyst are interspersed with flat foils carrying a predominantly platinum catalyst. During a thermal runaway methane burns as a hot plasma gas which releases hydrogen and CH₃ free radicals. If these can be quenched on the catalyst surface the thermal runaway may be halted. Platinum is more effective than palladium at quenching these free radicals, and therefore the provision of a predominantly platinum catalyst on a flat foil that is sandwiched between two corrugated foils is likely to reduce the incidence of thermal runaway.

It will also be appreciated that the catalyst must be selected taking into account the heat transfer capabilities of the reactor block 12, for example the greater the distance between one flat plate and the next in the stack (i.e. the height of the castellations), the less effective is the heat transfer; while the thermal conductivity of the material of the flat plates and castellated plates also affects the heat transfer rates. This heat transfer problem is more acute in channels in which the height exceeds the width of the channel, especially when the catalyst is provided on a stack of corrugated foils separated by flat foils as mentioned above.

In the course of operation of the reactor module 10 there will be a tendency for the catalysts in both the reforming channels 15 and the combustion channels 16 to degrade and become less effective. To some extent this may be compensated for, for example by increasing the temperatures to which the gas mixtures are preheated before they are introduced into each reactor block 12. If the pressure within the combustion channels 16 is increased the rate of combustion also increases; over the life of the catalyst it may therefore be advantageous to gradually increase pressure in order to maintain the same level of activity as the combustion catalyst degrades. A further variable is the partial pressure of oxygen in the combustion channels, in particular in the second reactor block 12b, and this may be modified by introducing an oxygen-rich gas instead of air through the inlet 24. This may be done throughout the life of the reactor module 10, or only as the catalyst degrades. Another variable is the fuel ratio between the first reactor block 12a and the second reactor block 12b; not only may this ratio be adjusted as discussed earlier, but the composition of the fuel introduced at inlet 26 to the second reactor block 12b may differ from that provided to the first reactor block 12a. For example in the context of Fischer-Tropsch synthesis the tail gas may be separated into a hydrogen-rich fraction and a hydrogen-poor fraction; hence the fuel supplied to the reactor blocks 12 can therefore be selected between methane, or the hydrogen-poor tail gas, or the hydrogen-rich fraction, which have different combustion properties, and the proportions of these different fuels may be varied during the operational lifetime of the reactor module 10.

It will nevertheless be appreciated that as the catalysts degrade, despite the adjustments and variations described above, it will become inevitable that the production rate of synthesis gas from the reactor module 10 will eventually decrease. If, as described above, a plant incorporates several such reaction modules 10 arranged in parallel, the plant capacity can be adjusted by changing the number of reaction modules that are utilised, by bringing online reaction modules 10 that had previously not been used. At some stage it would be necessary to remove and replace or refurbish reaction modules 10 where the catalyst has degraded excessively. Typically a reactor module 10 would be switched off and another reactor module 10 brought online to take its place; the switched-off reactor module 10 can be removed, and replaced by a new or refurbished reactor module 10. This enables the plant to operate at substantially constant capacity. The reactor module 10 that has been removed may either be scrapped, or may be refurbished by replacing the catalysts in the channels 15 and 16.

1.-13. (canceled)

14. A method of performing an endothermic reaction in a reaction module, the module comprising:
a plurality of separate reactor blocks, each reactor block defining a multiplicity of first and second flow channels arranged alternately within the block to ensure thermal contact between the first and second flow channels, with catalyst in the first flow channels for the endothermic reaction, and with catalyst in the second flow channels for a combustion reaction,
the reactor blocks being arranged and connected for series flow of a gas mixture to undergo the endothermic reaction in the first flow channels and also for flow of a combustible gas mixture in the second flow channels, such that the endothermic reaction mixture flows in series through the reactor block,
wherein the method comprises supplying the gas mixture to undergo the endothermic reaction to the first flow channels, and supplying the combustible gas mixture to the second flow channels, and during the course of operation of the reaction module modifying the reaction conditions in the second flow channels to compensate for degradation of the catalysts.

15. The method of claim 14 wherein gas mixtures are provided to the first and the second flow channels of the reactor blocks preheated to elevated temperatures, and the preheat temperatures are varied during the operation of the catalytic reaction module.

16. The method of claim 14 wherein fuel gases are supplied to the second flow channels of the reactor blocks, and the composition of the fuel gases that are supplied are varied during the operation of the catalytic reaction module.

17. The method of claim 14 wherein the combustion reaction is carried out in at least two reactor blocks in series, in the same sequence as the reactor blocks for the endothermic reaction, such that the combustion gas mixture emerging from one reactor block is subjected to treatment before it is introduced to the next reactor block, wherein the treatment involves the addition of an oxygen-rich gas.

18. The method of claim 14 wherein the combustion reactions are carried out at an elevated pressure, and the pressure is varied during operation of the catalytic reaction module.

19. The method of claim 14 wherein inert components are added to the gas mixture supplied to combustion channels of at least one of the reactor blocks.

20. A catalytic reaction module for performing an endothermic reaction, the module comprising a plurality of separate reactor blocks, each reactor block defining a multiplicity of first and second flow channels arranged alternately within the block to ensure thermal contact between the first and second flow channels, with catalyst in the first flow channels for the endothermic reaction, and with catalyst in the second flow channels for a combustion reaction, the reactor blocks being arranged and connected for series flow of a gas mixture to undergo the endothermic reaction in the first flow channels and also for flow of a combustible gas mixture in the second flow channels, such that the endothermic reaction mixture flows in series through the reactor block, wherein the first flow channels and/or the second flow channels are more than 2 mm wide in their narrowest transverse dimension, and the catalyst in the channel is provided on a stack of corrugated foils separated by substantially flat foils, wherein the nature of the catalyst on the flat foils differs from that on the corrugated foils.

21. The reaction module of claim 20 wherein the catalyst differs by virtue of changes in catalyst loading.

22. The reaction module of claim 20 wherein the catalyst differs by virtue of changes in the loading of the active catalytic material.

23. The reaction module of claim 20 wherein the catalyst differs in the nature of the catalytic material.

24. The reaction module of claim 20 wherein the second flow channels are more than 2 mm wide in their narrowest transverse dimension, and the catalyst in the second flow channels is provided on a stack of corrugated foils separated by substantially flat foils, in which the corrugated foils carry a predominantly palladium-based catalyst and the flat foils carry a predominantly platinum catalyst.
